(19) United States
(12) Patent Application Publication
(10) Pub. No.: US 2022/0031316 A1
(43) Pub. Date: Feb. 3, 2022

(54) BARBED SUTURE AND MANUFACTURING METHOD OF THE BARBED SUTURE

(71) Applicants: Kuo-Cheng CHANG, TOUFEN CITY (TW); Wen-Chieh CHEN, HSINCHU CITY (TW)

(72) Inventors: Kuo-Cheng CHANG, TOUFEN CITY (TW); Wen-Chieh CHEN, HSINCHU CITY (TW)

(21) Appl. No.: 16/942,950

(22) Filed: Jul. 30, 2020

(51) Int. Cl. A61B 17/06

(52) U.S. Cl. CPC A61B 17/06166; A61B 2017/00526; A61B 2017/06176; A61B 2017/06171

(57) ABSTRACT

The present invention is related to a suture having a cord member. The cord member includes a cord and an outer later covering the core. An outer circumferential surface of the core includes a plurality of protrusions, and the plurality of protrusions are arranged in a ring shape relative to a center of the core. Through spiral cutting method to perform processing on the outer circumferential surface of the outer surface, a plurality of cutting slots are formed at the outer circumferential surface of the outer layer. After the processing of each cutting slot is complete, a protruding member for retaining skin is formed correspondingly.

### BACKGROUND OF THE INVENTION

### 1. Field of the Invention

The present invention relates to a suture for plastic surgery, and in particular, to a suture and a manufacturing of the suture.

### 2. Description of the Related Art

Due to aging and the effect of gravity, skin aging phenomena, such as wrinkles, crow's fee or nasolabial folds etc., becomes more prominent. If the skin aging condition is not obvious, it can be adjusted through extensive care or beautification therapy. However, if the aging condition is relatively more prominent such that extensive skin sagging occurs, then it is necessary to improve such condition through face lift operation. Although traditional face lift operation is able to improve the sagging and slacking due to skin aging, the drawbacks of general anesthesia process, slow post-surgical recovery time and scars left after the surgery etc. are often concerned by the people such that they tend to become reluctant in receiving the surgical operation despite the positive outcome of face lift operation.

To overcome various drawbacks associated with the traditional face lift operation, presently, a known face lift technique is to embed one or a plurality of suture equipped with hooks in the Subcutaneous tissue via injection needle in order to allow the suture to retain sagging dermis, thereby achieving the face lift effect. Since the entire treatment process, in comparison to the traditional face lift operation, results in relatively smaller wound and shorter recovery time in addition to that the surgical process does not require general anesthesia and post-surgical scars can also be prevented greatly. Consequently, such surgical operation provides a safer method to potential patients.

With regard to traditional sutures, for example, U.S. Pat. No. 8,975,332 discloses a technique of using a knife to cut out a plurality of hooks at the outer circumferential surface of a suture via a slanted cutting method. Nevertheless, the contact surface between the hooks formed via such method and the dermis is extremely limited and the retaining effect is insufficient. U.S. Patent Publication No. 2013/0226234A1 discloses that a suture core is a knitted core; however, since the dimension of a suture is extremely small, the knitting technique for the internal suture core requires much higher cost and longer manufacturing time. Taiwan Patent No. I692344 discloses a suture comprising a core, an intermediate layer covering the core and an outer layer covering the intermediate layer. The intermediate layer is able to reduce the structural damage caused by the rapid changes of strength and elasticity due to material difference. Nevertheless, the outer circumferential surface of the intermediate layer is smooth such that the bonding strength between the intermediate layer and the outer layer is poor. Consequently, it is likely to cause twisting, leading to angular of depth errors, during the cutting of the outer layer. In addition, when such suture is embedded into the subcutaneous tissue, it is also likely to affect the tension of the entire structure; therefore, there is a need for improvement.

### SUMMARY OF THE INVENTION

A primary objective of the present invention is to provide a suture capable of increasing the contact surface between the suture and the skin, reducing the cutting angle of the knife and increasing the overall structural strength.

To achieve the aforementioned primary objective, the present invention provides a suture comprising a cord member a plurality of protruding members. The cord member includes a core and an outer layer covering the core. An outer circumferential surface of the core includes a plurality of protrusions, and the plurality of protrusions are arranged in a ring shape relative to a center of the core. An outer circumferential surface of the outer layer includes a plurality of cutting slots, the plurality of cutting slots are arranged spaced apart from each other along an axial direction of the cord member, and the plurality of cutting slots extend in a spiral shape relative to the axial direction of the cord member respectively. The plurality of protruding members are arranged to protrude on an outer circumferential surface of the cord member and each one of the protruding members is integrally connected to a root portion of one of the cutting slots of the cord member and extends in a spiral shape relative to the axial direction of the cord member. In addition, a helix angle of each one of the protruding members is equal to a helix angle of one of the cutting slots of the cord member. The two helix angles can be adjusted arbitrarily depending upon the actual needs in practice.

Preferably, a ratio of a depth of each one of the cutting slots to a diameter of the cord member is between 0.05˜0.5, a ratio of a length of each one of the cutting slots to a diameter of the cord member is between 0.2˜2, a cutting angle of each one of the cutting slots is between 5 degrees˜60 degrees, and a ratio of a distance between the two adjacent cutting slots to a diameter of the cord member is between 0.1˜10.

Preferably, each one of the protrusions extends in a linear shape or a spiral shape along an axial direction of the core.

Preferably, a cross sectional shape of each one of the protrusions is a curved shape or a polygonal shape, allowing the core to have a non-circular appearance. Consequently, the contact surface area between the core and the outer layer can be increased and the strength of the contact interface can be enhanced. During the manufacturing, it is also able to reduce the twisting and deformation of the material, and the cutting related angles and dimensions can be more properly controlled.

In addition, the present invention further provides a manufacturing of the aforementioned suture. First, a knife is used to cut into the outer layer of the cord member from the outer circumferential surface of the outer layer of the cord member at an inclination angle. Next, the cord member is moved along the axial direction of the cord member and the cord member is rotated about the axial direction of the cord member as a center thereof during the movement, thereby allowing the outer circumferential surface of the outer layer of the cord member to be cut out of one of the protrusions by the knife. Finally, the knife is removed, allowing the outer circumferential surface of the outer layer of the cord member to form one of the cutting slots at a cutting area of the knife. Subsequently, the cord member can be rotated to a predefined angle or rotate in a reverse direction at an identical angle depending upon the actual needs. Then, the aforementioned steps can be repeated to perform the subsequent cutting and processing.

Preferably, a first material is poured into a first channel of a mold in order to form the core, and a second material is poured into a second channel of the mold in order to form the outer layer. Extrusion formation method is performed on the first material and the second material in a third channel of the mold in order to form the cord member.

From the above, it can be understood that the suture of the present invention utilizes the plurality of protrusions to increase the bonding between the core and the outer layer, and the material twisting can be reduced during the spiral cutting. Furthermore, the plurality of protrusions are used to increase the contact surface with the skin, such that it is able to achieve the effects of reducing the cutting angle and increasing the structural strength.

The detailed structure, features, assembly of the suture and the manufacturing method for the suture or the method of use thereof provided by the present invention are described in the following embodiments and detailed description. Nevertheless, a person with ordinarily skilled in the art in the technical field of the present invention shall understand that such detailed description and the specific embodiments exemplarily described in the present invention are provided to illustrate the present invention only such that they shall not be used to limit the claim scope of the present invention.

### BRIEF DESCRIPTION OF THE DRAWINGS

FIG. 1 is an outer appearance perspective view of the suture of the first embodiment of the present invention;

FIG. 2 is a cross sectional view of the suture of the first embodiment of the present invention;

FIG. 3 is a perspective view of the core provided by the suture of the first embodiment of the present invention;

FIG. 4 is a cross sectional view of the suture of the second embodiment of the present invention;

FIG. 5 is a perspective view of the core provided by the suture of the second embodiment of the present invention;

FIG. 6 is a cross sectional view of the suture of the third embodiment of the present invention;

FIG. 7 is a perspective view of the core provided by the suture of the third embodiment of the present invention;

FIG. 8 is a perspective view of the core provided by the suture of the fourth embodiment of the present invention;

FIG. 9 is a top view of the suture of the first embodiment of the present invention;

FIG. 10 is an axial cross sectional view of the suture of the first embodiment of the present invention;

FIG. 11 is a partially enlarged view of the suture of the first embodiment of the present invention;

FIG. 12A shows a process step of the manufacturing process of the present invention;

FIG. 12B shows a process step of the manufacturing process of the present invention;

FIG. 13 is an entrance end view of the mold provided by the manufacturing process of the present invention; and

FIG. 14 is an exit end view of the mold provided by the manufacturing process of the present invention.

### DETAILED DESCRIPTION OF THE INVENTION

First, the applicant emphasizes that in this specification, including the embodiments described below and the content defined in the scope of the claims, direction related terms are described based on the directions illustrated in the drawings. Secondly, in the embodiments and drawings described in the following, identical component signs represent identical or similar components or structural features thereof.

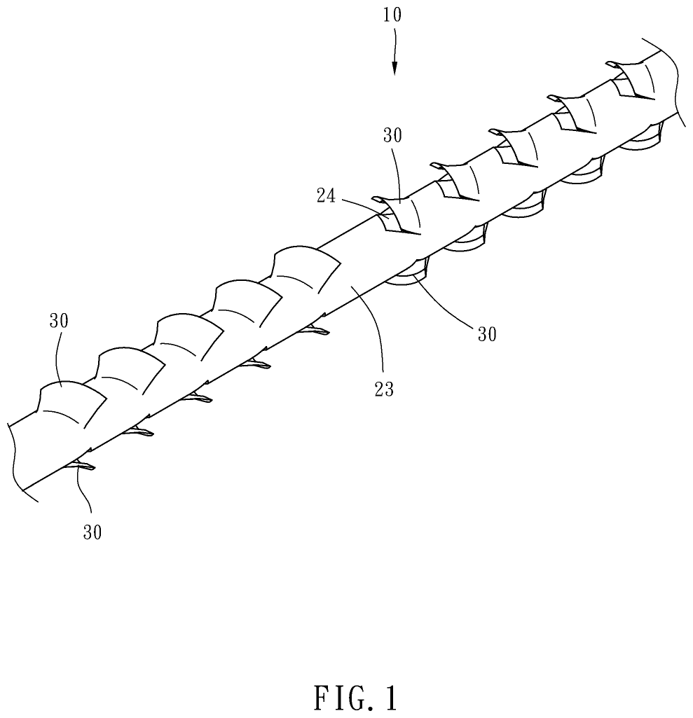

As shown in FIG. 1, a suture 10 of the present invention comprises a cord member 20 and a plurality of protruding members 30 (not limited to any specific quantity). To facilitate the explanation, a section of the suture 10 is illustrated in FIG. 1.

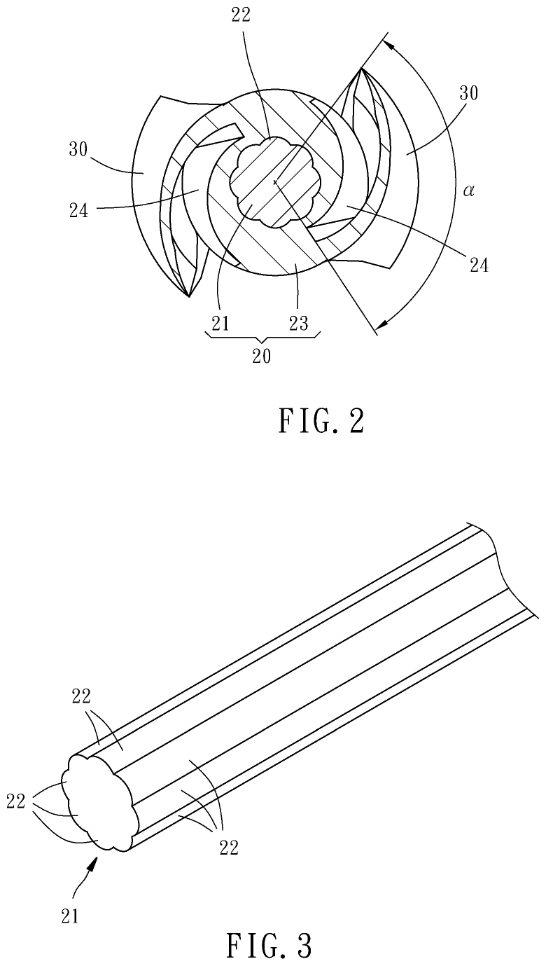

The cord member 20 includes a core 21 and an outer layer 23 covering the core 21. As shown in FIG. 2, the ratio of the core 21 to the diameter SD of the cord member is 0.2˜0.8. The ratio of the outer 23 to the diameter SD of the cord member 20 is 0.2˜0.8. For the materials of the core 21 and the outer layer 23, an absorbable material, such as plain catgut, chromic gut, Dexon, Vicryl, polycaprolactone (PCL), Polypropylene carbonate (PPC), polytrimethylene carbonate, polydioxanone (PDO), PGA, PLGA, LA or polydioxanone (PDS) etc., can be selected for use; or an nonabsorbable material, such as silk, cotton, nylon, polyester, polyethylene, polypropylene, polytetrafluoroethylene, stainless steel or polybutylene etc., can be selected for use. In this embodiment, the material of the core 21 is PGA, and the material of the outer layer 23 is LA. Both of the materials can be used in conjunction with each other depending upon the actual needs; however, regardless how the two are used in conjunction with each other, the tensile strength of the core 21 is greater than the tensile strength of the outer layer 23. In addition, the two can also be made of the same material but of different manufacturing criteria as long as there is a difference between the tensile strengths of the two. Furthermore, since the function of the outer layer 23 is to reduce wear and to increase the lubricity, the outer circumferential surface of the outer layer 23 can be coated with a layer of material promoting the skin repair, such as collagen.

As shown in FIG. 2 and FIG. 3, the outer circumferential surface of the core 21 includes a plurality of protrusions 22 (eight protrusions are shown here; however, the present invention is not limited to such number only). The plurality of protrusions 22 are arranged in a ring shape relative to the center of the core 21 and extend linearly along the axial direction of the core 21 respectively. In addition, the cross sectional shape of each protrusion 22 is a curved shape in this embodiment.

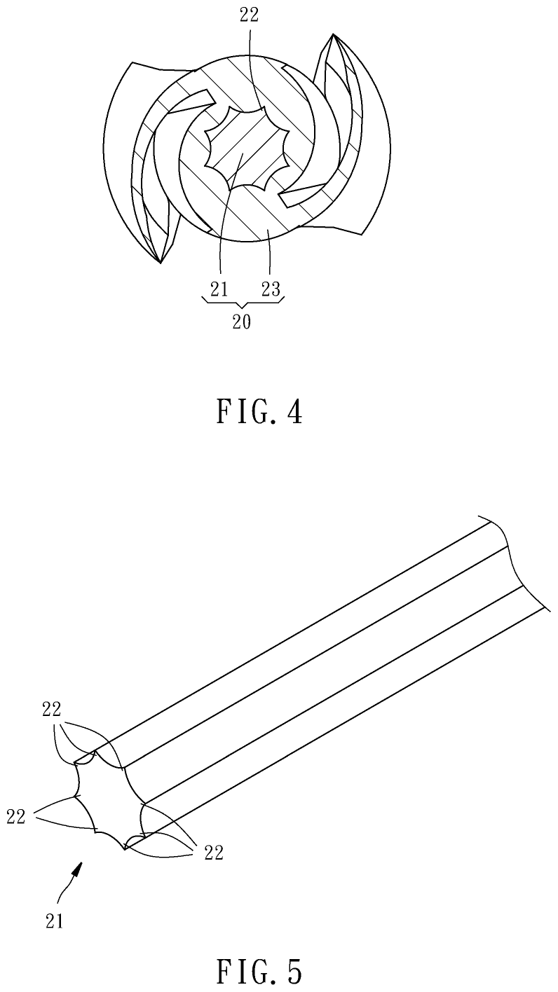

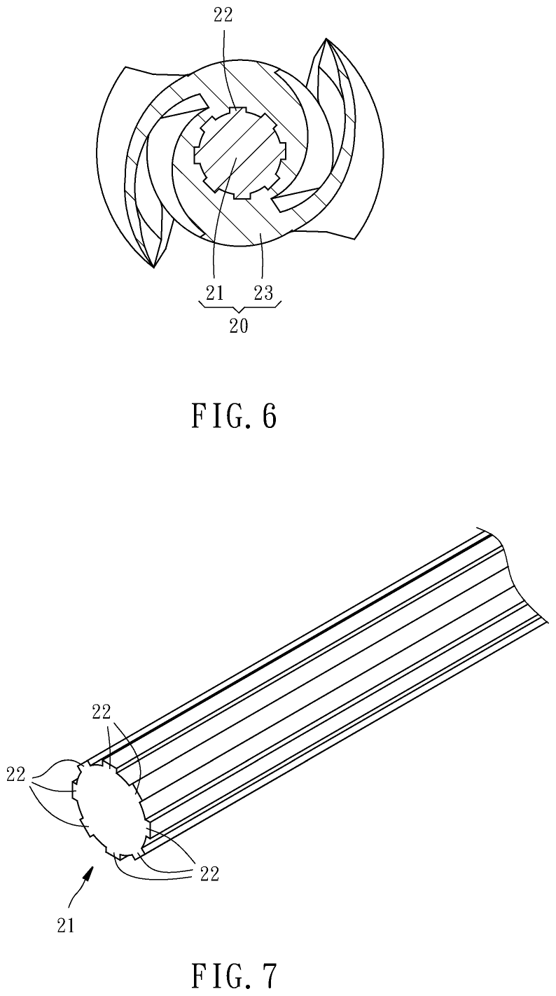

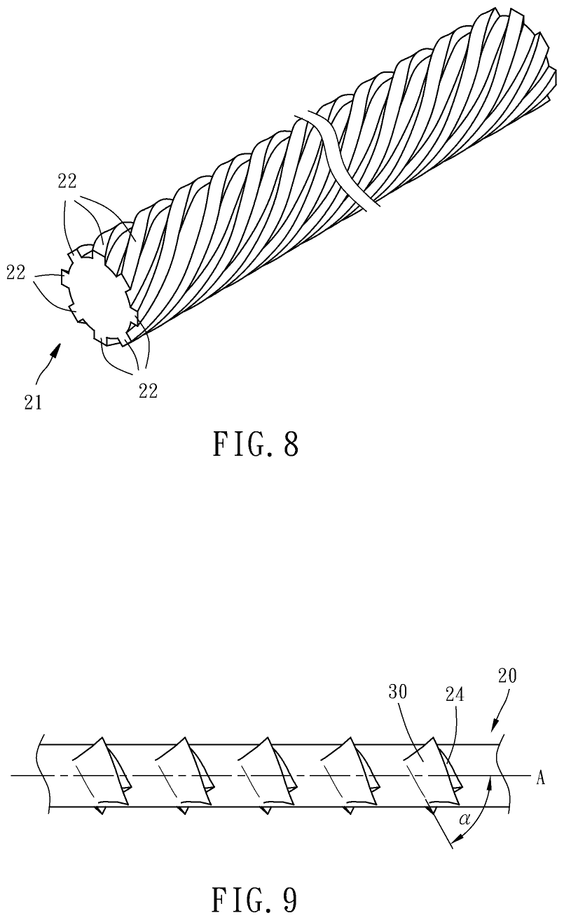

Furthermore, it shall be noted that the configuration of the protrusion 22 can have different variations. For example, the cross sectional shape of the protrusion 22 can be, such as, a curved triangular shape as shown in FIG. 4 or a rectangular shape as shown in FIG. 6. Moreover, the protrusion 22 can extend in a linear shape along the axial direction of the core 21 (as shown in FIG. 5 and FIG. 7), and it can also extend in a spiral shape along the axial direction of the core 21 (as shown in FIG. 8).

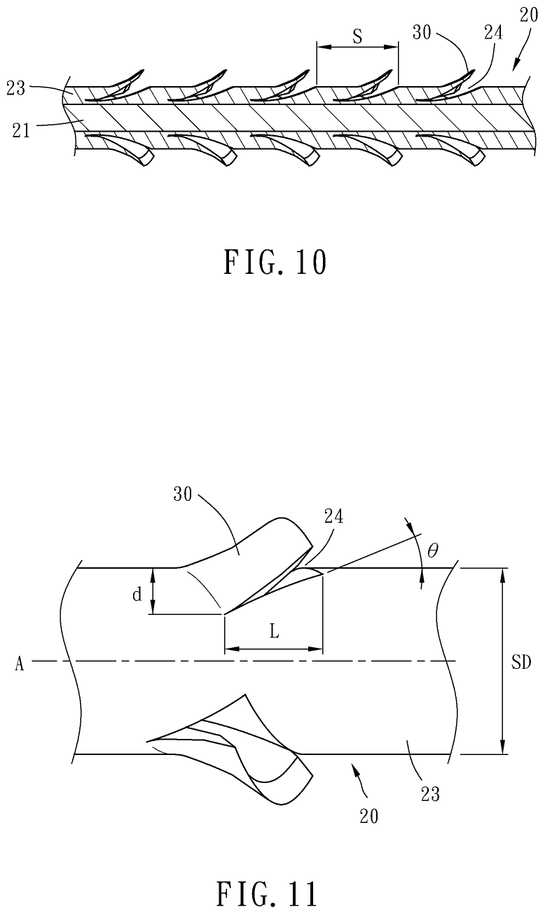

As shown in FIG. 9 to FIG. 11, the outer circumferential surface of the outer layer 23 includes a plurality cutting slots 24. The plurality of cutting slots 24 are arranged spaced apart from each other along the axial direction A of the cord member 20, and the plurality of cutting slots 24 extend in a spiral shape relative to the axial direction A of the cord member 20 respectively. In addition, a ratio of the depth d of the cutting slot 24 to the diameter SD of the cord member 20 is between 0.05˜0.5. A ratio of the length L of the cutting slot to the diameter SD of the cord member 20 is between 0.2˜2. A cutting angle θ of the cutting slot 24 is between 5˜60 degrees. A ratio of a distance S between the two adjacent cutting slots 24 to the diameter SD of the cord member 20 is between 0.1˜10.

In addition, it shall be noted that in this embodiment, the plurality of cutting slots 24 are arranged in pairs in order to be spaced apart from each other along the axial direction A of the cord member 20. In other words, each pair of cutting slots 24 are spaced apart from each other by 180 degrees. However, in practice, the plurality of cutting slots 24 arranged in a ring shape with the axial direction A of the cord member 20 as a center thereof can be adjusted between 0˜360 degrees depending upon the actual needs. Furthermore, the two adjacent cutting slots 24 can also be arranged to face toward each other or in an alternating manner.

The plurality of protruding members 30 protrude on the outer circumferential surface of the outer layer 23 of the cord member 20 and adopt the method of one-to-one to be integrally connected to the root portion 25 of the plurality of cutting slots 24 respectively. In addition, as shown in FIG. 9, the protruding members 30 and the cutting slots 24 have the same helix angle a. The aforementioned helix angle a in this embodiment is 60 degrees; however, in practice, the aforementioned helix angle a can be increased or decreased depending upon the actual needs.

In addition, the plurality of protruding members 30 are formed simultaneously during the process when the plurality of cutting slots 24 are cut out on the outer circumferential surface of the outer layer 23. To be more specific, the manufacturing method of the present invention mainly comprises the following steps:

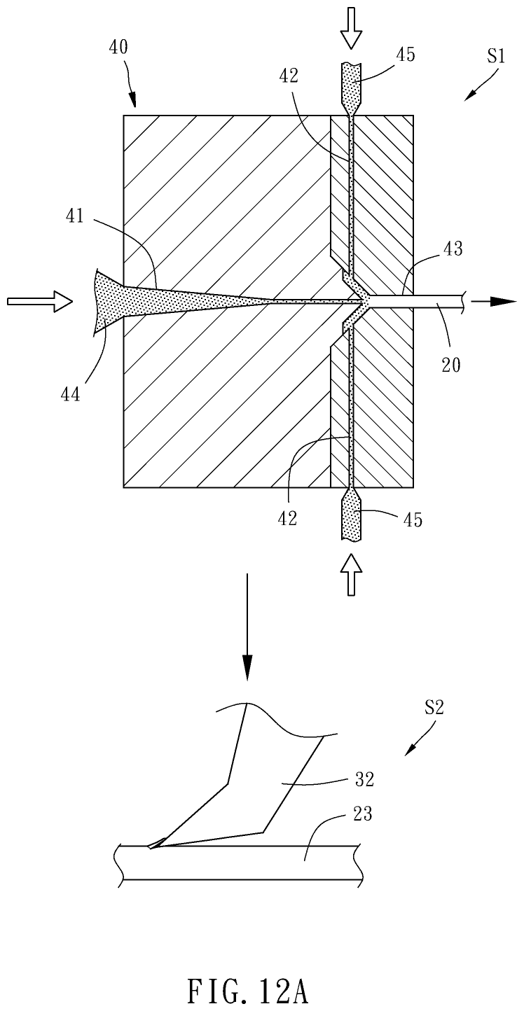

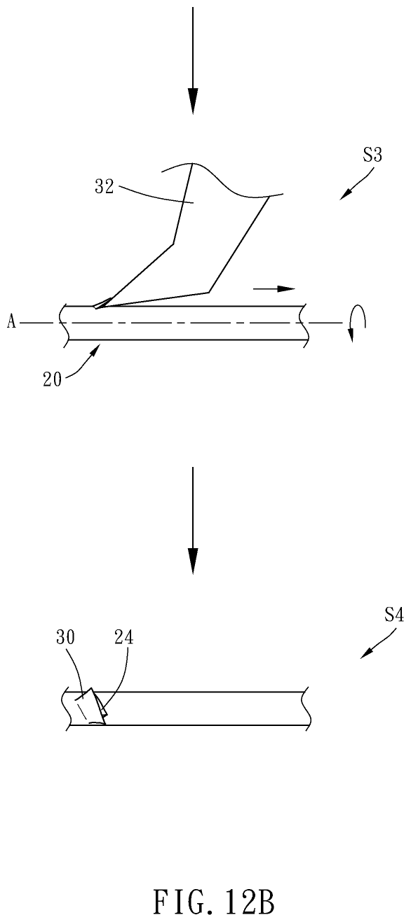

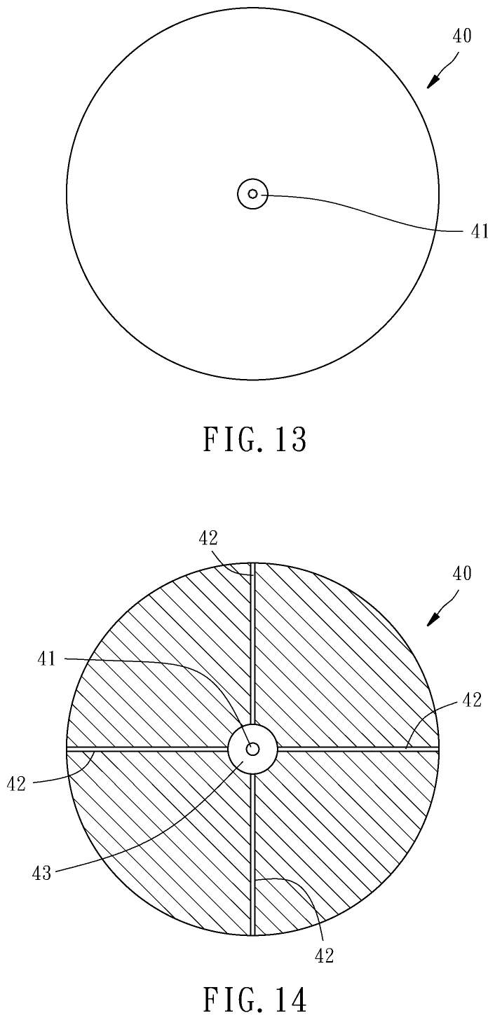

a) As shown in Step S2 of FIG. 12A, a knife 32 is used to cut into the outer layer 23 from the outer circumferential surface of the outer layer 23 at an inclination angle. However, before the knife 32 is used to perform spiral cutting on the outer layer 23, as shown in Step S1 of FIG. 12A, FIG. 13 and FIG. 14, a first material 44 for forming the core 21 is poured into a first channel 41 of a mold 40, and a second material 45 for forming the outer layer 23 is poured into a second channel 42 of the mold 40 respectively. An extrusion formation is performed on the first material 44 and the second material 45 in a third channel 43 of the mold 40 in order to form the cord member 20.

b) As shown in Step S3 of FIG. 12B, after the cutting depth is confirmed to not exceed the outer layer 23, the cord member 20 is moved along the axial direction A of the cord member 20 by a distance, and during the process of moving, the cord member 20 is rotated by a predefined angle about the axial direction A of the cord member 20 as a center thereof, thereby allowing the outer circumferential surface of the cord member 20 to be cut by the knife 32 to form a protruding member 30 extending in a spiral shape.

c) As shown in Step S4 of FIG. 12B, the knife 32 is removed, allowing the cutting area of the outer circumferential surface of the cord member 20 cut by the knife 32 to form a cutting slot 24 extending in a spiral shape.

After the first cutting slot 24 is formed completely, the cord member 20 can be subsequently moved or rotated depending upon the actual needs. For example, the cord member 20 can be moved based on a separation distance between two adjacent cutting slots 24 at the front or rear or the cord member 20 can be rotated by an angle between two vertical adjacent cutting slots 24 relative to the axis of the cord member 20, following which the aforementioned steps S1˜S4 can be repeated in order to complete the manufacturing of the suture 10.

In view of the above, the suture 10 of the present invention utilizes the plurality of protrusions 22 to increase the bonding between the core 21 and the outer layer 23. In addition, during the spiral cutting, material twisting can be reduced. Furthermore, the plurality of protruding members 30 are further utilized to increase the contact surface with the skin, and the cutting angle can be reduced, thereby allowing the spiral protruding members 30 to have a strong root portion 25 in order to achieve the effect of increasing the structural strength.

What is claimed is:

1. A suture, comprising:
a cord member having a core and an outer layer covering the core; an outer circumferential surface of the core having a plurality of protrusions, and the plurality of protrusions arranged in a ring shape relative to a center of the core; an outer circumferential surface of the outer layer having a plurality of cutting slots, the plurality of cutting slots arranged spaced apart from each other along an axial direction of the cord member, and the plurality of cutting slots extending in a spiral shape relative to the axial direction of the cord member respectively; and
a plurality of protruding members arranged to protrude on an outer circumferential surface of the cord member; each one of the protruding members integrally connected to a root portion of one of the cutting slots of the cord member and extending in a spiral shape relative to the axial direction of the cord member; a helix angle of each one of the protruding members being equal to a helix angle of one of the cutting slots of the cord member.

2. The suture according to claim 1, wherein a ratio of a depth of each one of the cutting slots to a diameter of the cord member is between 0.05˜0.5.

3. The suture according to claim 1, wherein a ratio of a length of each one of the cutting slots to a diameter of the cord member is between 0.2˜2.

4. The suture according to claim 1, wherein a cutting angle of each one of the cutting slots is between 5 degrees-60 degrees.

5. The suture according to claim 1, wherein a ratio of a distance between the two adjacent cutting slots to a diameter of the cord member is between 0.1˜10.

6. The suture according to claim 1, wherein the outer circumferential surface of the outer layer is coated with a material promoting a skin repair.

7. The suture according to claim 1, wherein a tensile strength of the core is greater than a tensile strength of the outer layer.

8. The suture according to claim 1, wherein the core and the outer layer are made of an absorbable material.

9. The suture according to claim 1, wherein the core is made of a nonabsorbable material, and the outer layer is made of an absorbable material.

10. The suture according to claim 1, wherein a ratio of the core over a diameter of the cord member is 0.2˜0.8; a ratio of the outer layer over a diameter of the cord member is 0.2˜0.8.

11. The suture according to claim 1, wherein each one of the protrusions extends in a linear shape along an axial direction of the core.

12. The suture according to claim 1, wherein each one of the protrusions extends in a spiral shape along an axial direction of the core.

13. The suture according to claim 1, wherein a cross sectional shape of each one of the protrusions is a curved shape or a polygonal shape.

14. A manufacturing method for the suture according to claim 1, comprising the following steps:
a) use a knife to cut into the outer layer of the cord member from the outer circumferential surface of the outer layer of the cord member at an inclination angle;
b) move the cord member along the axial direction of the cord member and rotate the cord member about the axial direction of the cord member as a center thereof during the movement, thereby allowing the outer circumferential surface of the outer layer of the cord member to be cut out of one of the protrusions by the knife; and
c) remove the knife, and allow the outer circumferential surface of the outer layer of the cord member to form one of the cutting slots at a cutting area of the knife.

15. The manufacturing method according claim 14, wherein before step a), pour a first material into a first channel of a mold in order to form the core, and pour a second material into a second channel of the mold in order to form the outer layer; perform extrusion formation method on the first material and the second material in a third channel of the mold in order to form the cord member.
